(19) United States
(12) Patent Application Publication
(10) Pub. No.: US 2021/0145474 A1
(43) Pub. Date: May 20, 2021

(54) COMPOSITE ATHERECTOMY BURR

(71) Applicant: BOSTON SCIENTIFIC SCIMED, INC., MAPLE GROVE, MN (US)

(72) Inventors: LASZLO TRENT FARAGO, HUDSON, WI (US); DAVID B. MORRIS, ANOKA, MN (US)

(73) Assignee: BOSTON SCIENTIFIC SCIMED, INC., MAPLE GROVE, MN (US)

(21) Appl. No.: 16/953,177

(22) Filed: Nov. 19, 2020

Related U.S. Application Data
(60) Provisional application No. 62/938,121, filed on Nov. 20, 2019.

(51) Int. Cl. A61B 17/3207

(52) U.S. Cl. CPC A61B 17/320758; A61B 2017/00398

(57) ABSTRACT

An atherectomy system includes a composite atherectomy burr including a first atherectomy burr component formed of a first material having a first material property and a second atherectomy burr component formed of a second material having a second material property, the second atherectomy burr component extending within and secured to the first atherectomy burr component, a drive mechanism adapted to rotatably actuate the composite atherectomy burr and a controller that is adapted to regulate operation of the drive mechanism. The second atherectomy burr component may be mechanically secured to the first atherectomy burr component.

### CROSS-REFERENCE TO RELATED APPLICATIONS

This application claims the benefit of priority under 35 U.S.C. § 119 to U.S. Provisional Application Ser. No. 62/938,121, filed Nov. 20, 2019, the entirety of which is incorporated herein by reference.

### TECHNICAL FIELD

The present disclosure pertains to medical devices, and methods for manufacturing and using medical devices. More particularly, the disclosure is directed to devices and methods for removing occlusive material from a body lumen. Further, the disclosure is directed to an atherectomy device for forming a passageway through an occlusion of a body lumen, such as a blood vessel.

### BACKGROUND

Many patients suffer from occluded arteries and other blood vessels which restrict blood flow. Occlusions can be partial occlusions that reduce blood flow through the occluded portion of a blood vessel or total occlusions (e.g., chronic total occlusions) that substantially block blood flow through the occluded blood vessel. In some cases a stent may be placed in the area of a treated occlusion. However, restenosis may occur in the stent, further occluding the vessel and restricting blood flow. Revascularization techniques include using a variety of devices to pass through the occlusion to create or enlarge an opening through the occlusion. Atherectomy is one technique in which a catheter having a cutting element thereon is advanced through the occlusion to form or enlarge a pathway through the occlusion. A need remains for alternative atherectomy devices to facilitate crossing an occlusion.

### SUMMARY

This disclosure provides design, material, manufacturing method, and use alternatives for medical devices. As an example, an atherectomy system includes a composite atherectomy burr including a first atherectomy burr component formed of a first material having a first material property and a second atherectomy burr component formed of a second material having a second material property, the second atherectomy burr component extending within and secured to the first atherectomy burr component. A drive mechanism is adapted to rotatably actuate the composite atherectomy burr and a controller is adapted to regulate operation of the drive mechanism.

Alternatively or additionally, the first material property may include hardness.

Alternatively or additionally, the first material property may include toughness.

Alternatively or additionally, the second material property may include weldability.

Alternatively or additionally, the second atherectomy burr component may be mechanically secured to the first atherectomy burr component.

Alternatively or additionally, the second atherectomy burr component may be press fitted onto the first atherectomy burr component.

Alternatively or additionally, the second atherectomy burr component may be soldered to the first atherectomy burr component.

Alternatively or additionally, the first material may have a first crystalline structure and the second material may have a second crystalline structure.

Alternatively or additionally, the first atherectomy burr component may include a cutting surface.

Alternatively or additionally, the drive mechanism may include a drive coil coupled with the composite atherectomy burr and a drive motor adapted to rotate the drive coil, wherein the second atherectomy burr component is adapted to be welded to the drive coil.

Alternatively or additionally, the drive mechanism and the composite atherectomy burr may be adapted to accommodate a guidewire extending therethrough.

Alternatively or additionally, the atherectomy system may further include a handle including a handle housing, the drive motor disposed within the handle housing.

Another example is a composite atherectomy burr adapted for use with an atherectomy system including a drive coil and a drive motor adapted to rotatably actuate the drive coil, the composite atherectomy burr adapted to be secured to the drive coil. The composite atherectomy burr includes a first atherectomy burr component having an inner surface defining an inner profile and an outer surface that is adapted to be machined into a cutting surface, and a second atherectomy burr component having an outer surface defining an outer profile that is complementary to the inner profile of the first atherectomy burr component. The second atherectomy burr component extends at least partially into the first atherectomy burr component.

Alternatively or additionally, the inner profile may include a first cylindrical portion having a first diameter and a second cylindrical portion having a second diameter different than the first diameter.

Alternatively or additionally, the inner profile may include a reduced diameter distal region, and the complementary outer profile of the second atherectomy burr component may extend into the reduced diameter distal region and forms an interference fit therewith.

Alternatively or additionally, the first atherectomy burr component may be formed of a material that is not weldable to the second atherectomy burr component.

Alternatively or additionally, the second atherectomy burr component may be mechanically secured to the first atherectomy burr component.

Another example is a composite atherectomy burr adapted for use with an atherectomy system including a drive coil and a drive motor adapted to rotatably actuate the drive coil, the composite atherectomy burr adapted to be secured to the drive coil. The composite atherectomy burr includes a first atherectomy burr component having an inner surface defining an inner profile and an outer surface that is adapted to be machined into a cutting surface, the inner profile including an initial solder reservoir and a first solder migration area, a second atherectomy burr component having an outer surface defining an outer profile that is complementary to the inner profile of the first atherectomy burr component, the outer profile including a second solder migration area, and solder that is initially disposed within the initial solder reservoir and adapted to reflow into the first solder migration area and the second solder migration area in order to secure the second atherectomy burr component to the first atherectomy burr component.

Alternatively or additionally, the second atherectomy burr component may extend at least partially into the first atherectomy burr component.

Alternatively or additionally, the composite atherectomy burr may have a finished overall length after solder reflow that is shorter than an initial overall length prior to solder reflow.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic block diagram of an example atherectomy system;

FIG. 2 is a schematic block diagram of an example atherectomy system;

FIG. 3 is a schematic block diagram of an example atherectomy system;

FIG. 4 is a schematic block diagram of an example atherectomy system;

FIG. 5 is a schematic block diagram of an example atherectomy system;

FIG. 6 is a perspective view of an example atherectomy system;

FIG. 7 is a perspective view of an example composite atherectomy burr useable in any of the example atherectomy systems of FIGS. 1 through 6;

FIG. 8 is an exploded perspective view of the example composite atherectomy burr of FIG. 7;

FIG. 9 is a side view of an example composite atherectomy burr useable in any of the example atherectomy systems of FIGS. 1 through 6;

FIG. 10 is a cross-sectional view of the example composite atherectomy burr of FIG. 9, taken along the line 10-10;

FIG. 11 is a schematic cross-sectional view of an example composite atherectomy burr useable in any of the example atherectomy systems of FIGS. 1 through 6;

FIG. 12 is a side view of an example composite atherectomy burr shown in a final configuration, the example composite atherectomy burr useable in any of the example atherectomy systems of FIGS. 1 through 6;

FIG. 13 is a schematic cross-sectional view showing a first atherectomy burr component and a second atherectomy burr component illustrated in an initial configuration prior to being moved into the final configuration shown in FIG. 12; and

FIG. 14 is a cross-sectional view of the example composite atherectomy burr of FIG. 12, taken along the line 14-14, with the first and second components illustrated in the final configuration.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Many patients suffer from occluded arteries, other blood vessels, and/or occluded ducts or other body lumens which may restrict bodily fluid (e.g. blood, bile, etc.) flow. Occlusions can be partial occlusions that reduce blood flow through the occluded portion of a blood vessel or total occlusions (e.g., chronic total occlusions) that substantially block blood flow through the occluded blood vessel. Revascularization techniques include using a variety of devices to pass through the occlusion to create or enlarge an opening through the occlusion. Atherectomy is one technique in which a catheter having a cutting element thereon is advanced through the occlusion to form or enlarge a pathway through the occlusion. Ideally, the cutting element excises the occlusion without damaging the surrounding vessel wall and/or a previously implanted stent where restenosis has occurred. However, in some instances the cutting element may be manipulated and/or advanced such that it contacts the vessel wall and/or the stent. Therefore, it may be desirable to utilize materials and/or design an atherectomy device that can excise an occlusion without damaging the surrounding vessel and/or a previously implanted stent where restenosis has occurred. Additionally, it may be desirable that a cutting element be useful in removing hard occlusive material, such as calcified material, as well as softer occlusive material. The methods and systems disclosed herein may be designed to overcome at least some of the limitations of previous atherectomy devices while effectively excising occlusive material. For example, some of the devices and methods disclosed herein may include cutting elements with unique cutting surface geometries and/or designs.

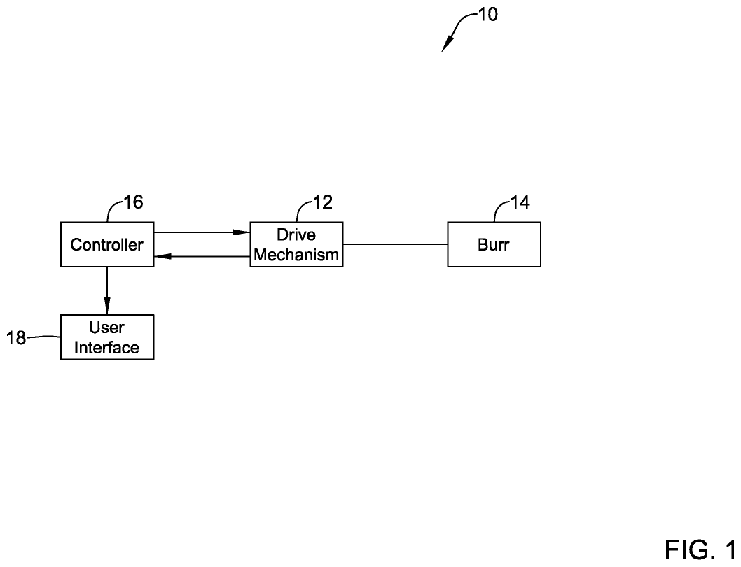

FIG. 1 is a schematic block diagram of an example atherectomy system 10 that includes a drive mechanism 12 that is adapted to rotatably actuate an atherectomy burr 14. While the example atherectomy system 10 is described herein as an electrically actuated atherectomy system, it will be appreciated that in some cases the atherectomy system 10 may instead be pneumatically actuated in which compressed air or another compressed fluid is used to drive a turbine that actuates the atherectomy burr 14. Illustrative but non-limiting examples of pneumatically actuated atherectomy systems include the Jetstream™ atherectomy system and the ROTABLATOR™ atherectomy systems available commercially from Boston Scientific Corporation.

The atherectomy system 10 includes a controller 16 that is adapted to regulate operation of the drive mechanism 12. In some cases, the atherectomy system 10 may include a user interface 18 that may be operably coupled to the controller 16 such that the controller 16 is able to display information regarding the performance of the drive mechanism 12. This information may, for example, include one or more of an instantaneous speed of the drive mechanism 12, an instantaneous torque being experienced by the atherectomy burr 14, and the like. In some instances, the atherectomy system 10 may not include the user interface 18. In some cases, the atherectomy burr 14 may also be referred to as being or including a cutting head or a cutting member, and these terms may be used interchangeably.

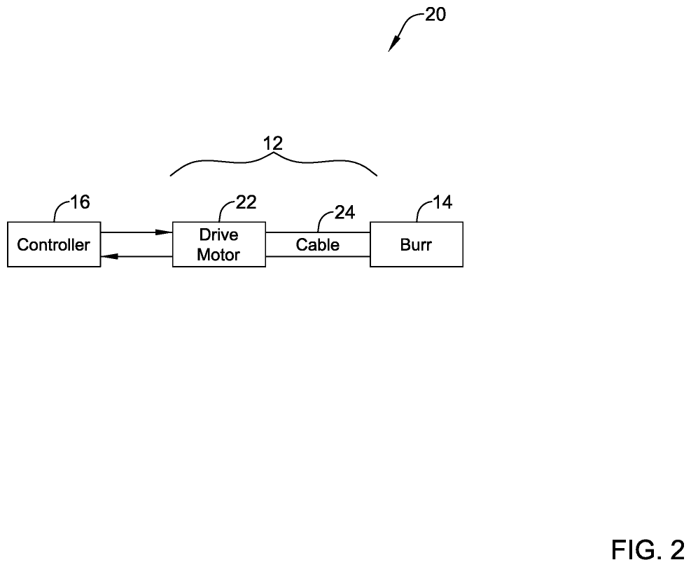

FIG. 2 is a schematic block diagram of an example atherectomy system 20 in which the drive mechanism 12 may include a drive motor 22 and a drive cable 24 that is operably coupled with the drive motor 22 as well as the atherectomy burr 14. In some cases, features of the atherectomy system 20 may be combined with features of the atherectomy system 10. In some cases, the atherectomy system 20 may also include a handle (not shown).

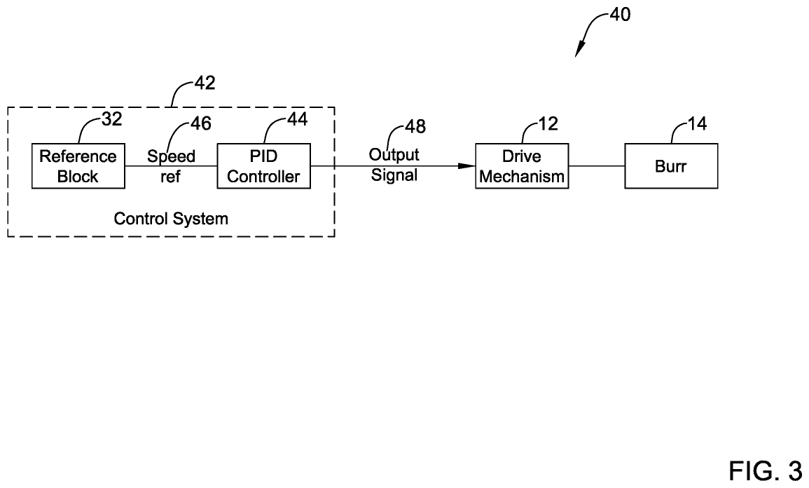

FIG. 3 is a schematic block diagram of an example atherectomy system 40 that includes a control system 42 that is adapted to regulate operation of the drive mechanism 12 in order to rotatably actuate the atherectomy burr 14. In some cases, features of the atherectomy system 40 may be combined with one or more of the atherectomy system 10 and the atherectomy system 20. The control system 42 may include a reference block 32 as well as a Proportional Integral Derivative (PID) controller 44 that is operably coupled to the reference block 32. In some cases, the reference block 32 may determine a speed reference 46 that is selectable between a nominal value, a negative value and zero. In some instances, the PID controller 44 may be further adapted to add an offset value to the speed reference 46 received from the reference block 32, thereby outputting an Output Signal 48, although in some cases, the reference block 32 may add the offset value.

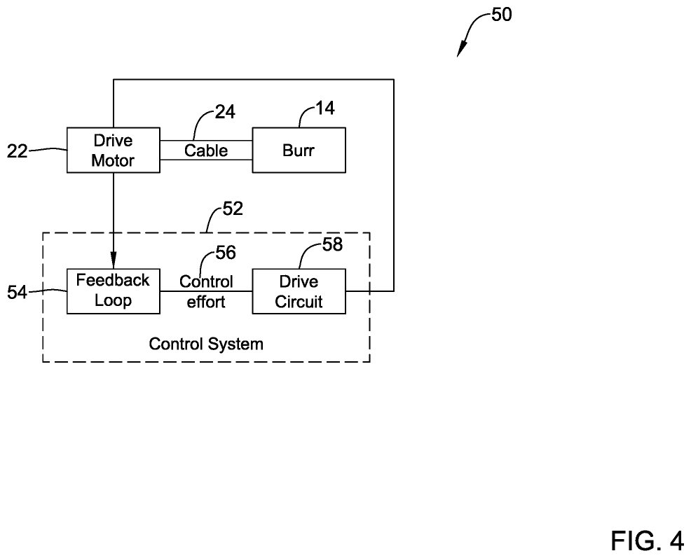

FIG. 4 is a schematic block diagram of an example atherectomy system 50 that includes a control system 52 that is adapted to regulate operation of the drive motor 22 in order to rotatably actuate the atherectomy burr 14. In some cases, features of the atherectomy system 50 may be combined with one or more of the atherectomy system 10, the atherectomy system 20 or the atherectomy system 40. The control system 52 is operably coupled to the drive motor 22 and includes a feedback loop 54 that is adapted to monitor performance of the drive motor 22 and to output a control effort signal 56. A drive circuit 58 is adapted to receive the control effort signal 56 and to regulate operation of the drive motor 22 in accordance with the control effort signal 56.

In some cases, the feedback loop 54 may include a reference block for determining a speed reference and a Proportional Integral Derivative (PID) controller that is operably coupled to the reference block for receiving the speed reference, the PID controller adapted to utilize the speed reference, a Proportional (P) gain value, an Integral (I) gain value and a Derivative (D) gain value in determining the control effort signal. In some cases, the feedback loop 54 may be adapted to add an offset value to a reference signal provided to the reference loop 54 in order to accurately hold speed of the drive motor 22 during a no-load situation. In some instances, for example if the atherectomy burr 14 becomes stuck, the control system 52 may be further adapted to increase the torque provided by the drive motor 22 until a torque threshold is reached for a brief period of time, and to subsequently direct the drive motor 22 to reverse at a slow speed in order to unwind energy in the drive mechanism.

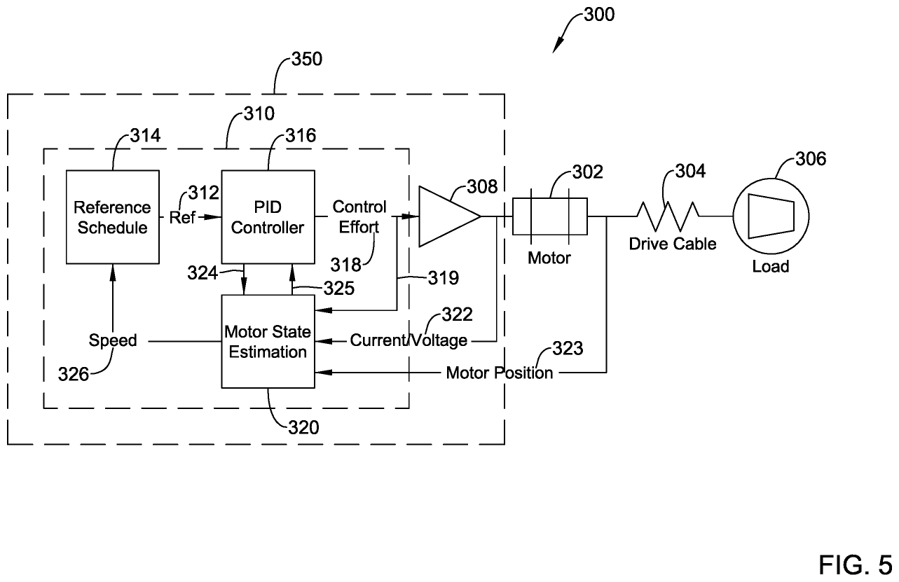

FIG. 5 is a schematic block diagram of an example atherectomy system 300. In some cases, the atherectomy system 300 may be considered as being an example of the atherectomy system 10, 20, 40 or 50. In some instances, features of the atherectomy system 300 may be combined with features of any of the atherectomy systems 10, 20, 40 or 50, for example. The atherectomy system 300 includes a motor 302 that drives a drive cable 304 which itself engages a load 306. The load 306 represents an atherectomy burr, for example. The motor 302 is controlled by a drive circuitry 308 which may be considered as being an example of or otherwise incorporated into the drive motor 22 (FIG. 2) and/or the controller 16 (FIGS. 1 and 2), for example. In some cases, the motor 302 may be sized, relative to the weight and other dimensions of the atherectomy system 300, to be capable of accelerating the atherectomy burr to full speed in less than 3 seconds, or in some cases in less than 2 seconds. As an example, the motor 302 may be rated for at least 60 watts. In a particular example, the motor 302 may be rated for about 80 watts. These are just examples.

The drive circuitry 308 receives an input from a feedback portion 310. In some cases, the feedback portion 310 begins with a reference input 312 from a reference schedule block 314, which provides the reference input 312 to a PID controller 316. In some cases, the reference schedule block 314 may be configured to accept additional inputs, such as from a user and/or from additional sensors not illustrated. As an example, if the device has been running for too long of a period of time, the reference schedule block 314 may reduce the speed reference in order to prevent overheating. A PID controller is a controller that includes a (P) proportional portion, an (I) integral portion and a (D) derivative portion. The PID controller 316 outputs a control effort value or reference current 318 to the drive circuitry 308. A motor state estimation block 320 receives a current/voltage signal 322 and a motor position signal 323 from the drive circuitry 308 and receives state feedback 324 from the PID controller 316. The motor state estimation block 320 provides a state feedback signal 325 back to the PID controller 316.

The motor state estimation block 320 outputs a speed value 326 back to the reference schedule block 314. While the feedback from the motor state estimation block 320 to the reference schedule block 314 is shown as being a speed value, in some cases the feedback may additionally or alternatively include one or more of position, torque, voltage or current, and in some cases may include the derivative or integral of any of these values. In some cases, the motor state estimation block 320 may instead receive a signal 323 that represents speed, instead of position (as illustrated). The motor position signal 323 may be an indication of relative rotational position of an output shaft of the motor 302, and thus an indication of relative rotational position of the load 306, which if tracked over time may provide an indication of speed.

In some cases, the drive circuitry 308 and the feedback loop 310 may in combination be considered as forming a controller 350 that is adapted to determine an estimated torque at the atherectomy burr (the load 306 as shown in FIG. 5). The controller 350 may be considered as being an example of the controller 16 (FIG. 1). In some cases, the controller 350 may be considered as including only some elements of the drive circuitry 308 and the feedback loop 310. In some instances, some of the features and functions of the controller 350 may take place in the motor state estimation block 320. It will be appreciated that while FIG. 5 shows various components as standalone components, in some cases the functions of one or more of the components may actually be spread between separate components. In some instances, the functions of one or more of the components may be combined into one or more components.

If the estimated torque at the load 306 becomes too high, this may be an indication that the burr is getting stuck. In order to protect against possible damage to the drive cable 304, and to protect against possible injury to the patient, the atherectomy system 300 may be adapted to stop or even reverse operation of the atherectomy system 300 if the estimated torque meets or exceeds a predetermined torque threshold. It will be appreciated that the actual value of the predetermined torque threshold may vary, depending on the mechanics of the atherectomy system 300, but may be set at a level low enough to prevent damage and injury, but not set so low as to engender too many false alarms caused by minor and/or temporary torque increases that are not caused by the load 306 becoming stuck. For example, the instantaneous torque may vary by small amounts as the atherectomy system 300 progresses through the patient's vasculature.

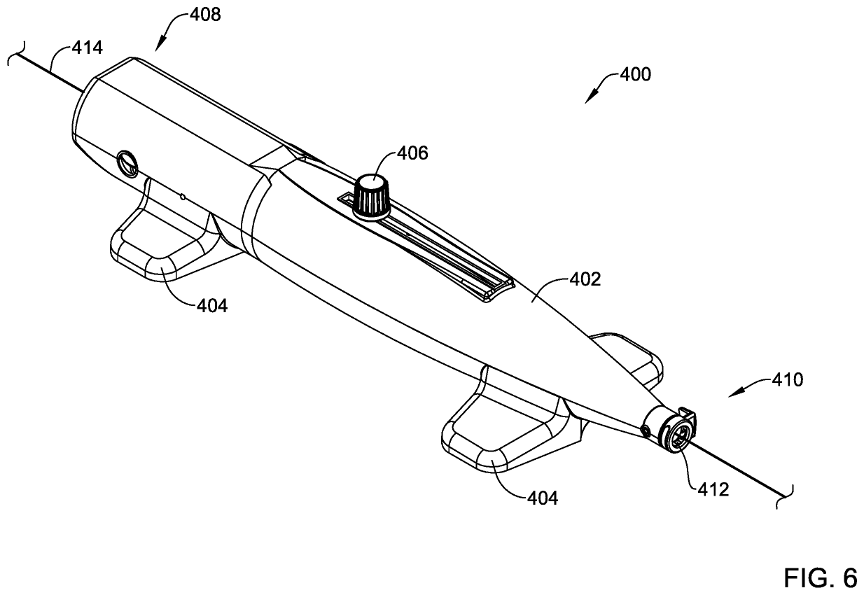

FIG. 6 is a perspective view of an example atherectomy system 400. In some cases, the atherectomy system 400 may be considered as being a manifestation of the atherectomy system 10, 20, 40, 50 or 300. In some instances, features of the atherectomy system 400 may be combined with features of any of the atherectomy systems 10, 20, 40, 50 or 300, for example. The atherectomy system 400 includes a handle 402. While not illustrated, it will be appreciated that the atherectomy system 400 includes a drive mechanism (such as the drive mechanism 12 shown in FIGS. 1-3) and a controller (such as the controller 16 shown in FIGS. 1-2) that is disposed within the handle 402 and that regulates operation of the drive mechanism. The handle 402 may, for example, include feet 404 that serve to stabilize the handle 402 on a flat surface during operation. A control mechanism 406 extends out of the handle 402 and may be used in controlling one or more features of the atherectomy system 400 during use. For example, the control mechanism 406 may be used to allow a user to change an operating speed of the drive mechanism.

The handle 402 includes a proximal region 408 and a distal region 410. As can be seen, the distal region 410 includes an aperture 412 that is adapted to permit a drive cable (such as the drive cable 24 of FIGS. 2 and 4 or the drive cable 304 of FIG. 5) to exit the handle 402. While not visible, the proximal region 408 may be configured to accommodate a guidewire 414 extending through the atherectomy system 400. It will be appreciated that at the distal region 410, the guidewire 414 will extend through the drive cable that is not shown in this illustration. In some cases, the atherectomy burr 14 may be attached to a simple drive mechanism (not illustrated) that does not include the controls discussed herein.

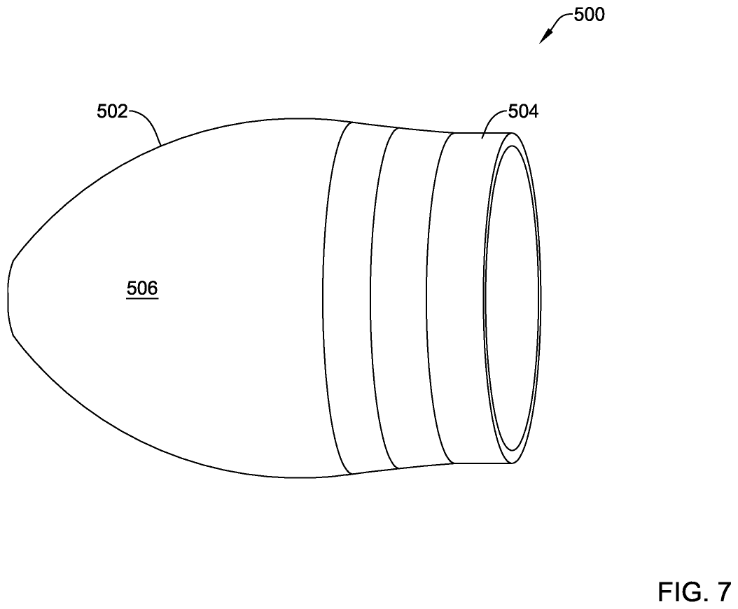

The atherectomy burr 14 (or the load 306, as shown in FIG. 5) may take a variety of forms. The atherectomy burr 14 may include any of a variety of different cutting patterns, as desired. In some instances, the atherectomy burr 14 may be considered as being a composite atherectomy burr that includes two (or more) distinct components that may be formed of different materials and may be joined together to form the composite atherectomy burr. For example, FIGS. 7 through 14 provide illustrative examples of composite atherectomy burrs. In some instances, for example, there may be a desire for one part of a composite atherectomy burr to have one or more particular properties and for another part of the composite atherectomy burr to have one or more properties that are different from those of the other part of the composite atherectomy burr. As an example, there may be a desire for a portion of the composite atherectomy burr that will ultimately form an atherectomy cutting surface to have a particular combination of hardness and toughness while another portion of the composite atherectomy burr, such as but not limited to an internal portion of the composite atherectomy burr, to be able to be easily welded to a drive coil.

In some instances, an outer portion of the composite atherectomy burr may be formed of a material that may or may not readily lend itself to attachment to other metals via welding. There are a number of materials, including some metals, that are not easily weldable. It will be appreciated that there may be a spectrum of weldability. Some materials may, for example, be weldable under strict conditions, but may not be very weldable outside of those strict conditions. Some materials may be weldable, but not very reproducibly or predictably. In some cases, for example, some metals have crystalline structures that can cause unpredictable cracking upon cooling. Stainless steel 440C is an example of a material that can undergo unpredictable cracking upon cooling. Some materials, including some metals simply are not weldable. Some metals are weldable to a first group of metals, but may not be easily weldable to certain other metals. It will be appreciated that there are combinations of metals that while otherwise weldable, are not easily weldable with each other. As an example, Metal A and metal B may each be weldable to other metals, but may not be easily weldable to each other. Other materials, such as composites, are not weldable.

Because there may be a desire to use non-weldable or poorly-weldable materials as the outer portion of the composite atherectomy burr, in some cases there may be a desire to provide a way to secure two (or more) components of the composite atherectomy burr in a manner that does not rely upon welding. In some cases, two (or more) components of the composite atherectomy burr may be combined using various mechanical techniques. For example, a first component may be frictionally secured to a second component. In some instances, a first component and a second component may be joined using an interference fit. A first component and a second component may, for example, be joined together using a third material that forms a mechanical interlock with each of the first component and a second component. An illustrative but non-limiting example of such a mechanical interlock may be the use of solder. In some cases, hard solder may be used to form a mechanical connection between a first component and a second component. In some instances, soft solder may be used to form a mechanical connection between a first component and a second component. In some cases, a first component and a second component may be joined in a mechanical connection via brazing. In some cases, welding may be considered as providing a mechanical connection between a first component and a second component. Other techniques are also contemplated.

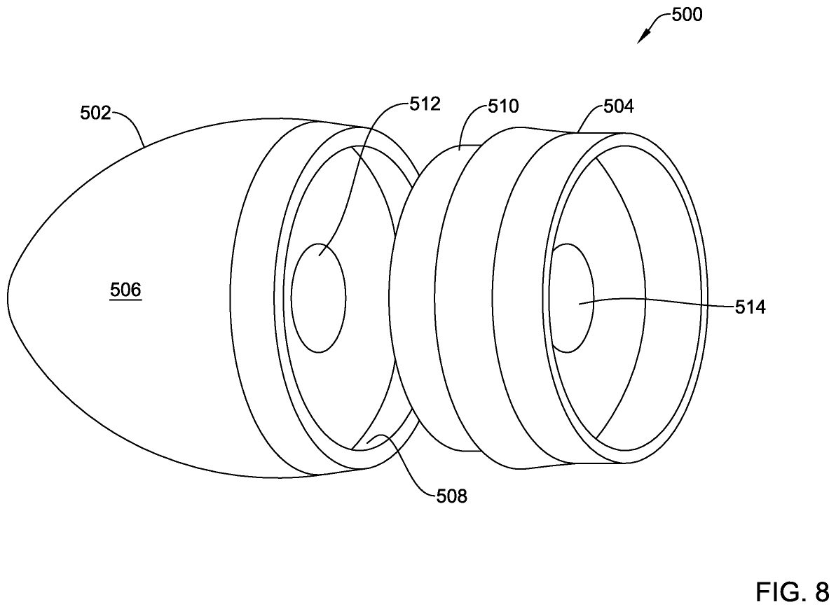

FIG. 7 is a perspective view of an example composite atherectomy burr 500 and FIG. 8 is an exploded perspective view of the composite atherectomy burr 500. As shown, the composite atherectomy burr 500 includes a first atherectomy burr component 502 and a second atherectomy burr component 504. In some cases, the first atherectomy burr component 502 may be considered as being formed of a first material having a first material property and the second atherectomy burr component 504 may be considered as being formed of a second material having a second material property. The first material and the second material may be completely different materials. The first material and the second material may both be the same metal, but with differing relative amounts of various additives, or with different crystalline structures, that provide the first material and the second material with differing properties.

As an example, the first material, which forms the first atherectomy burr component 502, may be selected to have a particular combination of hardness and toughness in order to provide an efficient, long-lasting cutting surface. While not shown, the cutting surface will be machined into an outer surface 506 of the first atherectomy burr component 502. The second material, which forms the second atherectomy burr component 504, may be selected to be easily secured to the drive coil of a drive mechanism (such as the cable 24 shown in FIGS. 2 and 4 or the drive cable 304 shown in FIG. 5). For example, the second material may be selected such that the second atherectomy burr component 504 may be easily welded to the drive cable. In some cases, for example, the first atherectomy burr component 502 may be formed of a material that is not weldable, or is poorly weldable, while the second atherectomy burr component 504 may be formed of a material that is weldable to other components, such as the drive cable.

As an illustrative but non-limiting example, the first material may have a first crystalline structure and the second material may have a second crystalline structure. In some cases, one of the first material and the second material may have a face-centered cubic (FCC) crystalline structure and the other of the first material and the second material may have a body-centered tetragonal (BCT) crystalline structure. In some instances, the first material and the second material may include a blend of martensitic steel and austenitic steel at a first ratio and the second material may include a blend of martensitic steel and austenitic steel at a second ratio. It will be appreciated that the ratios between martensitic steel and austenitic steel may each range from about 1 mass percent to about 99 mass percent (e.g., from about 1 mass percent martensitic steel and about 99 mass percent austenitic steel to about 99 mass percent martensitic steel and about 1 mass percent austenitic steel).

As seen in FIG. 8, the first atherectomy burr component 502 includes a first mating surface 508 while the second atherectomy burr component 504 includes a second mating surface 510 that is complementary to the first mating surface 508. While the second mating surface 510 is illustrated as fitting within the first mating surface 508, it will be appreciated that this is not necessary in all cases. The first atherectomy burr component 502 may be secured to the second atherectomy burr component 504 in any suitable manner. For example, there may be a mechanical connection such as but not limited to a press fit between the first atherectomy burr component 502 and the second atherectomy burr component 504. In some cases, the first atherectomy burr component 502 may be friction-weld to the second atherectomy burr component 504. Friction welding is a process whereby one of the components is held still while the other component is rapidly spun relative to the stationary component. The heat resulting from friction causes the two components to be welded to each other.

It can be seen that the first atherectomy burr component 502 includes an elongate aperture 512 and the second atherectomy burr component 504 includes an elongate aperture 514. In some cases, the elongate aperture 512 extends axially all the way through the first atherectomy burr component 502 and the elongate aperture 514 extends axially all the way through the second atherectomy burr component 504. Accordingly, the elongate aperture 512 and the elongate aperture 514 together form a guidewire aperture that extends through the composite atherectomy burr 500.

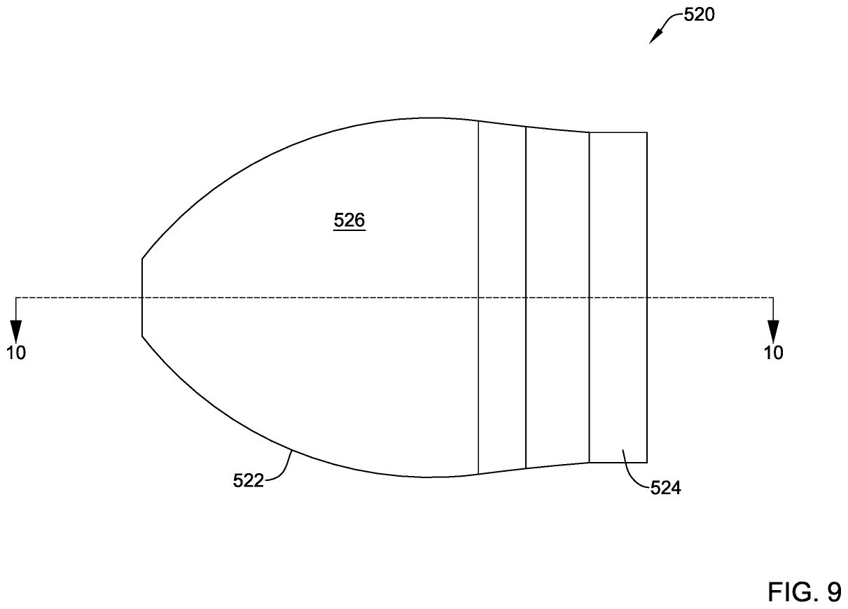

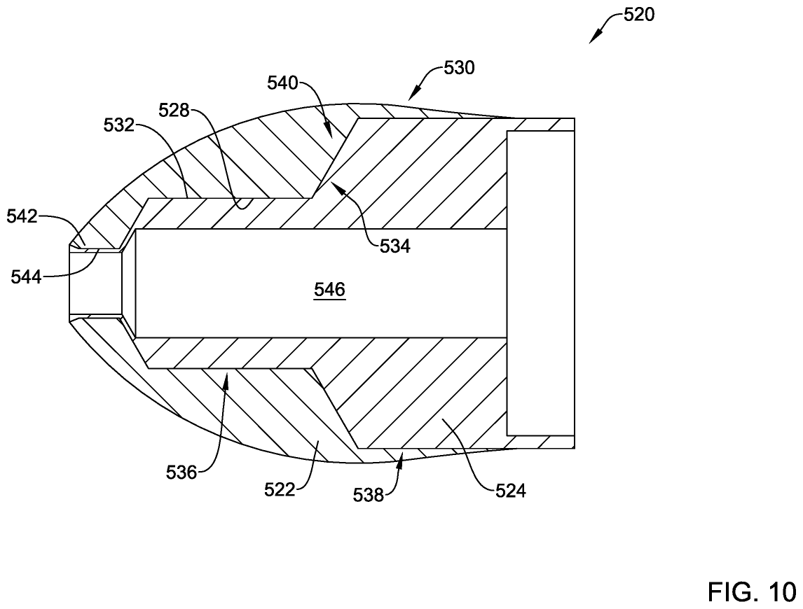

FIG. 9 is a side view of an example composite atherectomy burr 520 and FIG. 10 is a cross-sectional view of the composite atherectomy burr 520, taken along the line 10-10 in FIG. 9.

As shown, the composite atherectomy burr 520 includes a first atherectomy burr component 522 and a second atherectomy burr component 524. The first atherectomy burr component 522 includes an outer surface 526 that may be subsequently machined to provide a desired cutting surface. In some cases, the first atherectomy burr component 522 may be considered as being formed of a first material having a first material property and the second atherectomy burr component 524 may be considered as being formed of a second material having a second material property. The first material and the second material may be completely different materials. The first material and the second material may both be the same metal, but with differing relative amounts of various additives, or with different crystalline structures, that provide the first material and the second material with differing properties, as discussed above with respect to FIGS. 7 and 8.

As seen in FIG. 10, the first atherectomy burr component 522 includes an inner surface 528 defining an inner profile 530 while the second atherectomy burr component 524 includes an outer surface 532 defining an outer profile 534 that is complementary to the inner profile 530 of the first atherectomy burr component 522. In some cases, the inner profile 530 may include a first cylindrical portion 536 having a first diameter and a second cylindrical portion 538 having a second diameter different than the first diameter. As illustrated, there is a tapered portion 540 extending between the first cylindrical portion 536 and the second cylindrical portion 538. In some cases, there may instead be a step-wise change in diameter between the first cylindrical portion 536 and the second cylindrical portion 538. There may be a plurality of step-wise diameter changes in the inner profile 530 (with corresponding changes in the outer profile 534. The inner profile 530 may simply define a tapered shape. These are just examples.

The first atherectomy burr component 522 may be secured to the second atherectomy burr component 524 in any suitable manner. For example, there may be a mechanical connection such as but not limited to a press fit between the first atherectomy burr component 522 and the second atherectomy burr component 524. In some instances, as illustrated, the inner profile 530 may include a reduced diameter distal region 542 and the outer profile 534 may have a corresponding portion 544 that extends into the reduced diameter distal region 542 and may form an interference or swage fit therewith. As can be seen, a guidewire lumen 546 extends through the composite atherectomy burr 520.

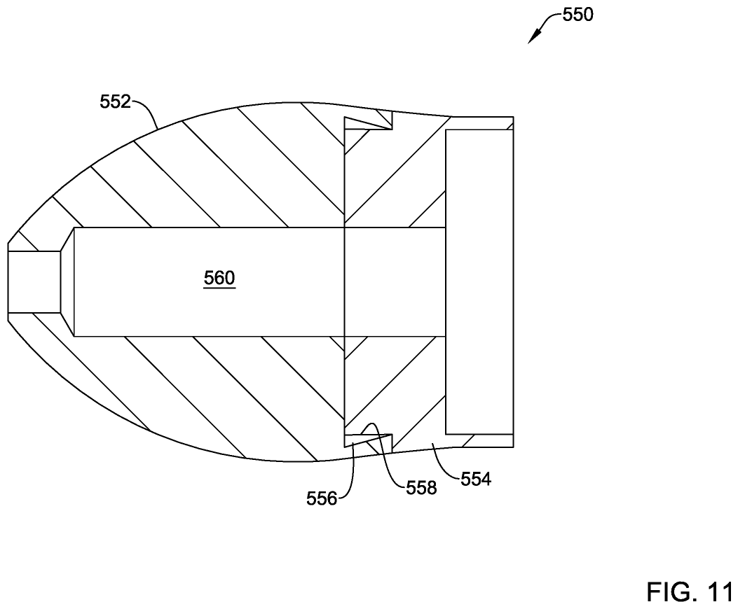

FIG. 11 is a schematic cross-sectional view of an example composite atherectomy burr 550 that includes a first atherectomy burr component 552 and a second atherectomy burr component 554. In some cases, the first atherectomy burr component 552 may be considered as being formed of a first material having a first material property and the second atherectomy burr component 554 may be considered as being formed of a second material having a second material property, as discussed above with respect to FIGS. 7 and 8. As shown in FIG. 11, the first atherectomy burr component 552 includes an engagement region 556 and the second atherectomy burr 554 includes a corresponding engagement region 558. The engagement region 556 may frictionally engage the corresponding engagement region 558 in order to secure the first atherectomy burr component 552 to the second atherectomy burr component 554. A guidewire lumen 560 may be seen as extending through the composite atherectomy burr 550.

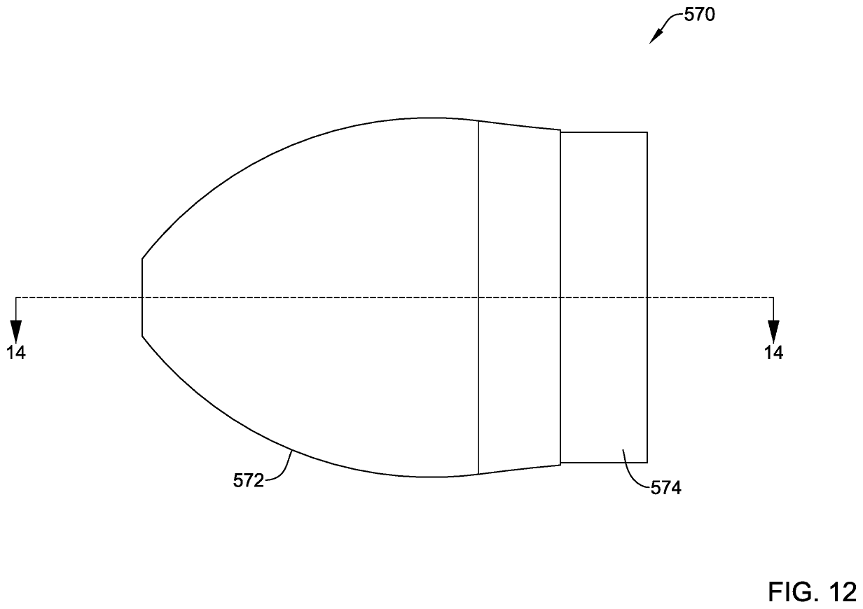

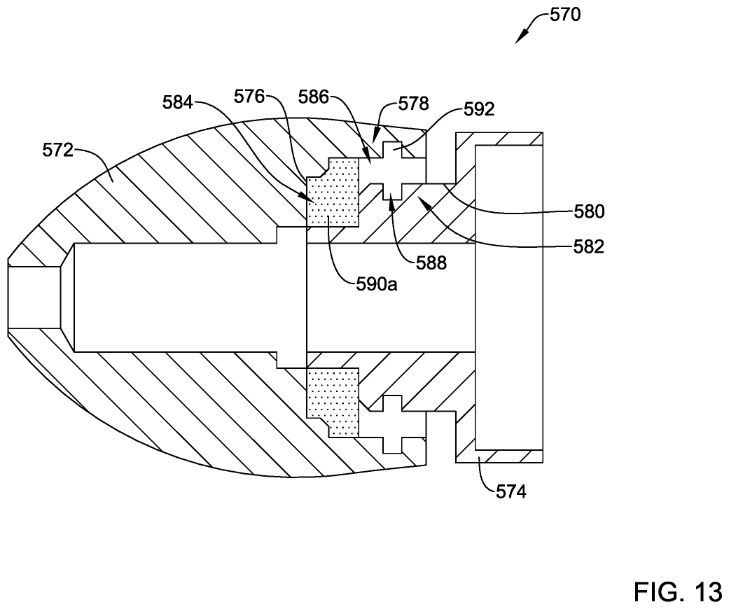

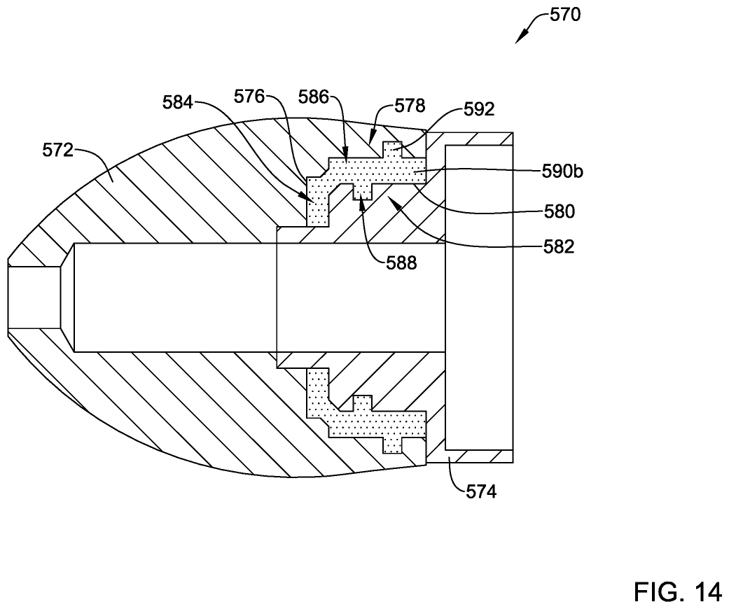

FIG. 12 is a side view of an example composite atherectomy burr 570. The composite atherectomy burr 570 includes a first atherectomy burr component 572 and a second atherectomy burr component 574. In some cases, the first atherectomy burr component 572 may be considered as being formed of a first material having a first material property and the second atherectomy burr component 574 may be considered as being formed of a second material having a second material property, as discussed above with respect to FIGS. 7 and 8. As will be discussed subsequently with respect to FIGS. 13 and 14, FIG. 12 shows the first atherectomy burr component 572 and the second atherectomy burr component 574 in a final configuration. FIG. 13 is a schematic cross-sectional view showing the first atherectomy burr component 572 and the second atherectomy burr component 574 in an initial configuration, while FIG. 14 is a cross-sectional view taken along the line 14-14 of FIG. 12, showing the first atherectomy burr component 572 and the second atherectomy burr component 574 in the final configuration (as shown in FIG. 12).

The first atherectomy burr component 572 includes an inner surface 576 defining an inner profile 578 while the second atherectomy burr component 574 includes an outer surface 580 defining an outer profile 582 that is complementary to the inner profile 578 of the first atherectomy burr component 572. The inner profile 578 includes an initial solder reservoir 584 that is adapted to initially hold a quantity of solder such as, but not limited to, silver solder. The inner profile 578 also includes a first solder migration area 586 that includes a notch 592. The outer profile 582 includes a second solder migration area 588 that forms a notch or recess. In order to secure the second atherectomy burr component 574 to the first atherectomy burr component 572, a quantity of solder may be placed within the initial solder reservoir 584, and the second atherectomy burr component 574 may be disposed within the first atherectomy burr component 572. Heat may be applied to cause the solder to reflow into the first solder migration area 586 (including the notch 592) and into the second solder migration area 588, indicated as solder 590b, thereby securing the second atherectomy burr component 574 to the first atherectomy burr component 572. It will be appreciated that the solder 590b extending into the notch 592 and into the second solder migration area 588 forms an interlock that secures the first atherectomy burr component 572 to the second atherectomy burr component 574.

As shown in FIG. 13, the second atherectomy burr component 574 may be seen as being axially spaced a short distance from the first atherectomy burr component 572. A solder material 590a is disposed within the initial solder reservoir 584. Comparing to FIG. 14, it can be seen that as the solder material 590a melts and reflows to form a reflowed solder material 590b, the second atherectomy burr component 574 may be seen as having moved axially closer to the first atherectomy burr component 572. The reflowed solder material 590b has now filled the first solder migration area 586 and the second solder migration area 586, thereby locking the first atherectomy burr component 572 to the second atherectomy burr component 574. It will be appreciated that the corresponding change in overall length of the composite atherectomy burr 570, from its initial configuration as shown in FIG. 13, and its final configuration as shown in FIGS. 12 and 14, may be used as an indicator that solder reflow occurred as intended, and that the first atherectomy burr component 572 is appropriately secured to the second atherectomy burr component 574.

The materials that can be used for the various components of the atherectomy burrs described herein may include a metal, metal alloy, polymer (some examples of which are disclosed below), a metal-polymer composite, ceramics, combinations thereof, and the like, or other suitable material. Suitable materials may, for example, include brass and various brass alloys. Alloys of copper and zinc may be used. Some examples of suitable metals and metal alloys include stainless steel, such as 304V, 304L, 316LV and 440C stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, radiopaque materials may be used to provide enhanced visibility during various imaging techniques. Radiopaque materials are understood to be materials capable of producing a relatively bright image on a fluoroscopy screen or another imaging technique during a medical procedure. This relatively bright image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

Various polymeric materials may be used. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some cases, coatings may be used. In these as well as in some other embodiments, a coating, for example a lubricious, a hydrophilic, a protective, or other type of coating may be used. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An atherectomy system, comprising:
a composite atherectomy burr including a first atherectomy burr component formed of a first material having a first material property and a second atherectomy burr component formed of a second material having a second material property, the second atherectomy burr component extending within and secured to the first atherectomy burr component;
a drive mechanism adapted to rotatably actuate the composite atherectomy burr; and
a controller adapted to regulate operation of the drive mechanism.

2. The atherectomy system of claim 1, wherein the first material property comprises hardness.

3. The atherectomy system of claim 1, wherein the first material property comprises toughness.

4. The atherectomy system of claim 1, wherein the second material property comprises weldability.

5. The atherectomy system of claim 1, wherein the second atherectomy burr component is mechanically secured to the first atherectomy burr component.

6. The atherectomy system of claim 1, wherein the second atherectomy burr component is press fitted onto the first atherectomy burr component.

7. The atherectomy system of claim 1, wherein the second atherectomy burr component is soldered to the first atherectomy burr component.

8. The atherectomy system of claim 1, wherein the first material has a first crystalline structure and the second material has a second crystalline structure.

9. The atherectomy system of claim 1, wherein the first atherectomy burr component comprises a cutting surface.

10. The atherectomy system of claim 9, wherein the drive mechanism comprises a drive coil coupled with the composite atherectomy burr and a drive motor adapted to rotate the drive coil; and
wherein the second atherectomy burr component is adapted to be welded to the drive coil.

11. The atherectomy system of claim 9, wherein the drive mechanism and the composite atherectomy burr are adapted to accommodate a guidewire extending therethrough.

12. The atherectomy system of claim 10, further comprising a handle including a handle housing, the drive motor disposed within the handle housing.

13. A composite atherectomy burr adapted for use with an atherectomy system including a drive coil and a drive motor adapted to rotatably actuate the drive coil, the composite atherectomy burr adapted to be secured to the drive coil, the composite atherectomy burr comprising:
a first atherectomy burr component having an inner surface defining an inner profile and an outer surface that is adapted to be machined into a cutting surface; and
a second atherectomy burr component having an outer surface defining an outer profile that is complementary to the inner profile of the first atherectomy burr component;
wherein the second atherectomy burr component extends at least partially into the first atherectomy burr component.

14. The composite atherectomy burr of claim 13, wherein the inner profile comprises a first cylindrical portion having a first diameter and a second cylindrical portion having a second diameter different than the first diameter.

15. The composite atherectomy burr of claim 14, wherein the inner profile includes a reduced diameter distal region, and the complementary outer profile of the second atherectomy burr component extends into the reduced diameter distal region and forms an interference fit therewith.

16. The composite atherectomy burr of claim 13, wherein the first atherectomy burr component is formed of a material that is not weldable to the second atherectomy burr component.

17. The composite atherectomy burr of claim 14, wherein the second atherectomy burr component is mechanically secured to the first atherectomy burr component.

18. A composite atherectomy burr adapted for use with an atherectomy system including a drive coil and a drive motor adapted to rotatably actuate the drive coil, the composite atherectomy burr adapted to be secured to the drive coil, the composite atherectomy burr comprising:
a first atherectomy burr component having an inner surface defining an inner profile and an outer surface that is adapted to be machined into a cutting surface, the inner profile including an initial solder reservoir and a first solder migration area;
a second atherectomy burr component having an outer surface defining an outer profile that is complementary to the inner profile of the first atherectomy burr component, the outer profile including a second solder migration area; and
solder initially disposed within the initial solder reservoir and adapted to reflow into the first solder migration area and the second solder migration area in order to secure the second atherectomy burr component to the first atherectomy burr component.

19. The composite atherectomy burr of claim 18, wherein the second atherectomy burr component extends at least partially into the first atherectomy burr component.

20. The composite atherectomy burr of claim 18, wherein the composite atherectomy burr has a finished overall length after solder reflow that is shorter than an initial overall length prior to solder reflow.
